(19) United States
(12) Patent Application Publication
(10) Pub. No.: US 2014/0263219 A1
(43) Pub. Date: Sep. 18, 2014

(54) LASER OPTICS WITH LATERAL AND ANGULAR SHIFT COMPENSATION

(71) Applicant: ABBOT CARDIOVASCULAR SYSTEMS INC., Santa Clara, CA (US)

(72) Inventor: Li Chen, San Jose, CA (US)

(73) Assignee: ABBOTT CARDIOVASCULAR SYSTEMS INC., Santa Clara, CA (US)

(21) Appl. No.: 14/209,665

(22) Filed: Mar. 13, 2014

Related U.S. Application Data
(60) Provisional application No. 61/798,651, filed on Mar. 15, 2013.

(51) Int. Cl. B23K 26/02; A61F 2/91

(52) U.S. Cl. CPC B23K 26/026; A61F 2/91
USPC 219/121.72; 219/121.67

(57) ABSTRACT

A telecentric F-theta lens is added to the optical chain of a laser used to cut stent patterns into a stent tube to facilitate positioning and alignment of the laser beam and to compensate for lateral and angular shift of the beam spot.

### CROSS-REFERENCES TO RELATED APPLICATIONS

This application claims priority from U.S. Application No. 61/798,651, filed Mar. 15, 2013 incorporated by reference in its entirety.

### BACKGROUND

1. Field of the Invention

The present invention relates generally to implantable medical devices and to a method for manufacturing implantable medical devices. These implantable medical devices may also be capable of retaining therapeutic materials and dispensing the therapeutic materials to a desired location of a patient's body. More particularly, the present invention relates to a system and method for forming the structure of a stent or intravascular or intraductal medical device, and IS particularly related to a combination of optical components used to compensate for lateral and angular shift of a laser beam used to form the stent structure from a tube.

2. General Background and State of the Art

In a typical percutaneous transluminal coronary angioplasty (PTCA) for compressing lesion plaque against the artery wall to dilate the artery lumen, a guiding catheter is percutaneously introduced into the cardiovascular system of a patient through the brachial or femoral arteries and advanced through the vasculature until the distal end is in the ostium. A dilatation catheter having a balloon on the distal end is introduced through the catheter. The catheter is first advanced into the patient's coronary vasculature until the dilatation balloon is properly positioned across the lesion.

Once in position across the lesion, a flexible, expandable, preformed balloon is inflated to a predetermined size at relatively high pressures to radially compress the atherosclerotic plaque of the lesion against the inside of the artery wall and thereby dilate the lumen of the artery. The balloon is then deflated to a small profile, so that the dilatation catheter can be withdrawn from the patient's vasculature and blood flow resumed through the dilated artery. While this procedure is typical, it is not the only method used in angioplasty.

In angioplasty procedures of the kind referenced above, restenosis of the artery often develops which may require another angioplasty procedure, a surgical bypass operation, or some method of repairing or strengthening the area. To reduce the likelihood of the development of restenosis and strengthen the area, a physician can implant an intravascular prosthesis, typically called a stent, for maintaining vascular patency. In general, stents are small, cylindrical devices whose structure serves to create or maintain an unobstructed opening within a lumen. The stents are typically made of, for example, stainless steel, nitinol, or other materials and are delivered to the target site via a balloon catheter. Although the stents are effective in opening the stenotic lumen, the foreign material and structure of the stents themselves may exacerbate the occurrence of restenosis or thrombosis.

A variety of devices are known in the art for use as stents, including expandable tubular members, in a variety of patterns, that are able to be crimped onto a balloon catheter, and expanded after being positioned intraluminally on the balloon catheter, and that retain their expanded form. Typically, the stent is loaded and crimped onto the balloon portion of the catheter, and advanced to a location inside the artery at the lesion. The stent is then expanded to a larger diameter, by the balloon portion of the catheter, to implant the stent in the artery at the lesion. Typical stents and stent delivery systems are more fully disclosed in U.S. Pat. No. 5,514,154 (Lau et al.), U.S. Pat. No. 5,507,768 (Lau et al.), and U.S. Pat. No. 5,569,295 (Lam et al.).

Stents are commonly designed for long-term implantation within the body lumen. Some stents are designed for non-permanent implantation within the body lumen. By way of example, several stent devices and methods can be found in commonly assigned and common owned U.S. Pat. No. 5,002,560 (Machold et al.), U.S. Pat. No. 5,180,368 (Garrison), and U.S. Pat. No. 5,263,963 (Garrison et al.).

Intravascular or intraductal implantation of a stent generally involves advancing the stent on a balloon catheter or a similar device to the designated vessel/duct site, properly positioning the stent at the vessel/duct site, and deploying the stent by inflating the balloon which then expands the stent radially against the wall of the vessel/duct. Proper positioning of the stent requires precise placement of the stent at the vessel/duct site to be treated. Visualizing the position and expansion of the stent within a vessel/duct area is usually done using a fluoroscopic or x-ray imaging system.

Although PTCA and related procedures aid in alleviating intraluminal constrictions, such constrictions or blockages reoccur in many cases. The cause of these recurring obstructions, termed restenosis, is due to the body's immune system responding to the trauma of the surgical procedure. As a result, the PTCA procedure may need to be repeated to repair the damaged lumen.

In addition to providing physical support to passageways, stents are also used to carry therapeutic substances for local delivery of the substances to the damaged vasculature. For example, anticoagulants, antiplatelets, and cytostatic agents are substances commonly delivered from stents and are used to prevent thrombosis of the coronary lumen, to inhibit development of restenosis, and to reduce post-angioplasty proliferation of the vascular tissue, respectively. The therapeutic substances are typically either impregnated into the stent or carried in a polymer that coats the stent. The therapeutic substances are released from the stent or polymer once it has been implanted in the vessel.

In the past, stents have been manufactured in a variety of manners, including cutting a pattern into a tube that is then finished to form the stent. The pattern can be cut into the tube using various methods known in the art, including using a laser.

Laser cutting of the stent pattern initially utilized lasers such as the Nd:YAG laser, configured either at its fundamental mode and frequency, or where the frequency of the laser light was doubled, tripled or even quadrupled to give a light beam having a desired characteristic to ensure faster and cleaner cuts.

Recently, lasers other than conventional Nd:YAG lasers have been used, such as diode-pumped solid-state lasers that operate in the short pulse pico-second and femto-second domains. These lasers provide improved cutting accuracy, but cut more slowly than conventional lasers such as the long pulse Nd:YAG laser.

A typical stent laser cutting system includes a laser source that directs a laser beam toward a stent tube through an optical chain. This optical chain may include intermediate components such as a minor and a lens, each of which is mechanically isolated from the stent tubing.

Ideally, the laser beam is directed toward the stent tube at approximately the top-dead-center position of the stent tube, as shown. In this case, the cutting spot of the laser coincides with the position that the cutting program anticipates the beam to be, resulting in the desired stent pattern. However, it is not uncommon for the laser beam to shift slightly away from this position. This can happen frequently because the large number of components in the optical chain such as mirrors, lenses, filters, and the stent tubing itself, need only move slightly to cause a shift in the beam path. When this occurs, there are two primary resulting defects. First, the cutting spot differs from the anticipated position and so the cut stent pattern may differ from the programmed pattern, which may cause thicker or thinner struts. Second, the beam may become defocused at the surface of the stent tube due to change in position of the cutting spot.

Additionally, because the beam is directed through the stent tubing at an angle, the resulting strut walls may not be perpendicular to the outer surface, which can cause the inner or outer strut width to differ from the desired dimension and result in variable stent strength. Since accurately cutting a stent pattern is of primary importance in achieving a product with the desired performance characteristics, there is a need for a laser cutting optical chain that will direct the laser beam perpendicular to stent tubing.

Furthermore, even if the laser beam is directed perpendicular to the cutting surface, there is currently no method of quickly and effectively checking the beam position and alignment or of adjusting those characteristics. The typical process for such realignment and repositioning requires a time consuming sequence of manually checking and observing the beam position and independently adjusting various components of the optical chain until the necessary beam position and alignment is achieved. The current process requires a stage-assistant alignment between the laser beam and stent tube that is costly and inefficient. This process introduces significant manufacturing inefficiencies that negatively impact the efficiency of stent manufacturing.

What has been needed, and heretofore unavailable, is an efficient and cost-effective laser cutting system that incorporates various features designed to sense and enhance the cutting performance and adjustability of the laser optical chain to ensure that the laser cutting spot is located at the a desired locations, such as the top dead center of the stent tube, as expected by the software controlling the cutting process. The present invention satisfies these, and other needs.

### SUMMARY OF THE INVENTION

In its most general aspect, the invention includes a laser cutting system using a telecentric F-theta lens as part of a laser optical chain to eliminate the need for time consuming alignment procedures during the stent cutting and manufacture of medical stents. In its various aspects, the invention introduces a substantial degree of efficiency into the cutting process improving the efficiency of the cutting process in a manner particularly important when employing short pulse lasers such as picosecond lasers that typically take longer to cut stents than traditional laser technologies.

In another general aspect, the invention includes a system and method for detecting the position of a laser beam relative to the surface of a stent tube into which the laser is cutting a stent pattern, and for repositioning and aligning the laser beam so as to improve the efficiency and accuracy of the cutting process.

In another aspect, the invention includes a laser system for cutting a stent pattern into a stent tube, comprising: a laser for generating a laser beam; a mirror for reflecting the laser beam at a desired angle; and a telecentric F-theta lens configured to receive the reflected laser beam and to focus the reflected laser beam onto a stent tube. In an alternative aspect, the system further comprises a partially reflecting and partially transmitting minor disposed in the laser beam between the minor and the telecentric F-theta lens; a first detector disposed to receive a portion of light from the incoming laser beam reflected by the partially reflecting and partially transmitting minor, the first detector configured to provide a signal representative of the position of the incoming laser beam on the first detector; a second detector disposed to receive a portion of light from a laser beam reflected from the stent tube and subsequently reflected by the partially reflecting and partially transmitting minor, the second detector configured to provide a signal representative of the position of the reflected laser beam on the second detector.

In still another alternative aspect, the system also may include a reflective surface disposed between the telecentric F-theta lens and the stent tube.

In another aspect, the invention may include a parallel plate disposed in the path of the incoming laser beam before the partially reflecting and partially transmitting mirror. In yet another aspect, the invention may include a Risley prism pair disposed in the path of the incoming laser beam before the partially reflecting and partially transmitting mirror.

In still another aspect, the present invention includes a method for aligning the focal spot of a laser beam used to cut a stent pattern into a stent tube, comprising: disposing a tiltable minor into a laser beam between a laser and a stent tube; disposing a telecentric F-theta lens into the laser beam between the tiltable mirror and the stent tube; focusing the laser beam to form a focused laser spot on the stent tube; and tiling the minor to adjust the position of the focused laser spot on the stent tube.

In yet another aspect, the present invention includes a method for compensating for lateral or angular shift of a laser beam focused on a stent tube, comprising: disposing a partially reflecting and partially transmitting minor between a laser generating a laser beam and focusing lens, the focusing lens configured to focus the laser beam at a location on the stent tube; disposing a first detector in a position to receive a portion of light from the incoming laser beam reflected by the partially reflecting and partially transmitting mirror, the first detector configured to provide a signal representative of the position of the incoming laser beam on the first detector; disposing a second detector in a position to receive a portion of light from a laser beam reflected from the stent tube and subsequently reflected by the partially reflecting and partially transmitting minor, the second detector configured to provide a signal representative of the position of the reflected laser beam on the second detector; and comparing the signals representing the positions of the incoming laser beam and the reflected laser beam to determine if the position of the incoming laser beam on the stent tube has changed.

In an alternative aspect, the method may include disposing a parallel plate into the laser beam prior to the partially reflecting and partially transmitting mirror; and arranging the parallel plate to laterally shift the position of the incoming laser beam on the stent tube.

In another alternative aspect, the method may include disposing a Risley prism pair into the laser beam prior to the partially reflecting and partially transmitting mirror; and arranging the Risley prism pair to shift the angular position of the incoming laser beam on the stent tube.

In still another alternative aspect, the method may include disposing a parallel plate into the laser beam after the Risley prism pair and before the partially reflecting and partially transmitting minor; and arranging the parallel plate to compensate a lateral shift in the position of the incoming laser beam introduced by the Risley prism pair.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a partial view of a stent showing various elements of a stent pattern.

FIG. 1A is a cross-sectional view of a portion of one of the elements of the stent pattern.

FIG. 2 is a side view of a typical arrangement of a computer controlled cutting station for cutting stent patterns into suitable tubing using a laser beam.

FIG. 3 is a schematic drawing illustrating an ideal set-up where a laser beam is focused at the top dead center position on a target, such as a stent tube.

FIG. 4 is a schematic drawing illustrating the effect on laser beam positioning at the target when one or more of the optical components in the laser optical chain is mal-positioned or misaligned relative to the target.

FIG. 5 is a schematic drawing illustrating one embodiment of the present invention using a telecentric F-theta lens.

FIG. 6 is a schematic drawing illustrating the effect on the position of the focal plane of a laser beam passing through a typical lens as a function of the entrance angle of the laser beam.

FIG. 7 is a schematic drawing illustrating the effect on the position of the focal plane of a laser beam passing through an F-theta lens as a function of the entrance angle of the laser beam.

FIG. 8 is a schematic drawing illustrating the effect on the position of the focal plane of a laser beam passing through a telecentric F-theta lens as a function of the entrance angle of the laser beam.

FIG. 9 is a schematic drawing illustrating an embodiment of a system for calibrating and aligning the position of a laser beam relative to a target, such as a stent tube.

### DETAILED DESCRIPTION OF THE PREFERRED EMBODIMENTS

FIG. 1 is an enlarged perspective view of a stent 10 illustrating an exemplary stent pattern and showing the placement of interconnecting elements 15 between adjacent radially expandable cylindrical elements. Each pair of the interconnecting elements 15 on one side of a cylindrical element are preferably placed to achieve maximum flexibility for a stent. In the embodiment shown in FIG. 1, the stent 10 has three interconnecting elements 15 between adjacent radially expandable cylindrical elements which are 120 degrees apart. Each pair of interconnecting elements 15 on one side of a cylindrical element are offset radially 60 degrees from the pair on the other side of the cylindrical element. The alternation of the interconnecting elements results in a stent which is longitudinally flexible in essentially all directions. Various configurations for the placement of interconnecting elements are possible. However, as previously mentioned, all of the interconnecting elements of an individual stent should be secured to either the peaks or valleys of the undulating structural elements in order to prevent shortening of the stent during the expansion thereof.

The number of undulations may also be varied to accommodate placement of interconnecting elements 15, for example, at the peaks of the undulations or along the sides of the undulations as shown in FIG. 1.

As best observed in FIG. 1, cylindrical elements in this exemplary embodiment are shown in the form of a serpentine pattern. As previously mentioned, each cylindrical element is connected by interconnecting elements 15. The serpentine pattern is made up of a plurality of U-shaped members 20, W-shaped members 25, and Y-shaped members 30, each having a different radius so that expansion forces are more evenly distributed over the various members.

The afore-described illustrative stent 10 and similar stent structures can be made in many ways. However, the preferred method of making the stent is to cut a thin-walled tubular member, such as, for example, stainless steel tubing to remove portions of the tubing in the desired pattern for the stent, leaving relatively untouched the portions of the metallic tubing which are to form the stent. In accordance with the invention, it is preferred to cut the tubing in the desired pattern by means of a machine-controlled laser, as exemplified schematically in FIG. 2.

The tubing may be made of suitable biocompatible material such as, for example, stainless steel. The stainless steel tube may be Alloy type: 316L SS, Special Chemistry per ASTM F138-92 or ASTM F139-92 grade 2. Special Chemistry of type 316L per ASTM F138-92 or ASTM F139-92 Stainless Steel for Surgical Implants. Other biomaterials may also be used, such as various biocompatible polymers, co-polymers or suitable metals, alloys or composites that are capable of being cut by a laser.

Another example of materials that can be used for forming stents is disclosed within U.S. application Ser. No. 12/070,646, the subject matter of which is intended to be incorporated herein in its entirety, which application discloses a high strength, low modulus metal alloy comprising the following elements: (a) between about 0.1 and 70 weight percent Niobium, (b) between about 0.1 and 30 weight percent in total of at least one element selected from the group consisting of Tungsten, Zirconium and Molybdenum, (c) up to 5 weight percent in total of at least one element selected from the group consisting of Hafnium, Rhenium and Lanthanides, in particular Cerium, (d) and a balance of Tantalum

The alloy provides for a uniform beta structure, which is uniform and corrosion resistant, and has the ability for conversion oxidation or nitridization surface hardening of a medical implant or device formed from the alloy. The tungsten content of such an alloy is preferably between 0.1 and 15 weight percent, the zirconium content is preferably between 0.1 and 10 weight percent, The molybdenum content is preferably between 0.1 and 20 weight percent and the niobium content is preferably between 5 and 25 weight percent.

The stent diameter is very small, so the tubing from which it is made must necessarily also have a small diameter. Typically the stent has an outer diameter on the order of about 0.06 inch in the unexpanded condition, the same outer diameter of the tubing from which it is made, and can be expanded to an outer diameter of 0.1 inch or more. The wall thickness of the tubing is about 0.003 inch or less.

Referring now to FIG. 2, the tubing 50 is put in a rotatable collet fixture 55 of a machine-controlled apparatus 60 for positioning the tubing 50 relative to a laser 65. According to machine-encoded instructions, the tubing 50 is rotated and moved longitudinally relative to the laser 65 which is also machine-controlled. The laser selectively removes the material from the tubing and a pattern is cut into the tube. The tube is therefore cut into the discrete pattern of the finished stent.

The process of cutting a pattern for the stent into the tubing is automated except for loading and unloading the length of tubing. Referring again to FIG. 2, it may be done, for example, using a CNC-opposing collet fixture 55 for axial rotation of the length of tubing, in conjunction with a CNC X/Y table 70 to move the length of tubing axially relatively to a machine-controlled laser as described. Alternatively, the collet fixture may hold the tube at only one end, leaving the opposite end unsupported. The entire space between collets can be patterned using the laser. The program for control of the apparatus is dependent on the particular configuration used and the pattern to be cut by the laser.

Referring now to FIG. 3, a typical stent laser cutting system 100 includes a laser source that directs a laser beam 105 toward a stent tube 120 through an optical chain. This optical chain may include intermediate components such as a mirror 110 and a lens 115, each of which is mechanically isolated from the stent tubing.

Ideally, the laser beam is directed toward the stent tube at approximately the top-dead-center position 125 of the stent tube, as shown. In this case, the cutting spot of the laser coincides with the position that the cutting program anticipates the beam to be, resulting in the desired stent pattern. However, it is not uncommon for the laser beam to shift slightly away from this position, as shown in FIG. 4. This can happen frequently because the large number of components in the optical chain such as mirrors, lenses, and the stent tubing itself, need only move slightly to cause a shift in the beam path. When this occurs, there are two primary resulting defects. First, the cutting spot 130 (FIG. 4) differs from the anticipated position and so the cut stent pattern may differ from the programmed pattern, which may cause thicker or thinner struts. Second, the beam may become defocused at the surface of the stent tube due to change in position of the cutting spot.

Additionally, because the beam is directed through the stent tubing at an angle, the resulting strut walls may not be perpendicular to the outer surface, which can cause the inner or outer strut width to differ from the desired dimension and result in variable stent strength. Since accurately cutting a stent pattern is of primary importance in achieving a product with the desired performance characteristics, there is a need for a laser cutting optical chain that will direct the laser beam perpendicular to stent tubing.

In one embodiment, the system uses a telecentric F-theta lens as part of a laser optical chain to ensure that the laser beam is directed perpendicular to the stent tubing. This mitigates the need for time consuming alignment procedures for the stent cutting process, and introduces a substantial degree of efficiency into the cutting process, which is particularly important when employing short pulse lasers such as picosecond lasers that naturally take longer to cut stents than traditional laser technologies.

FIG. 5 is a schematic drawing illustrating a laser system 200 into which a telecentric F-theta lens 215 has been introduced into the optical chain. Use of such a telecentric F-theta lens provides for correcting misalignment of the laser beam by simply adjusting the angulation of a minor 210. A primary advantage of using a telecentric F-theta lens is that regardless of the angle at which the laser beam is directed into the telecentric F-theta lens, it will exit the lens at the same angle. The positioning of the beam, however, changes with the change in angle of entry of the laser beam. Therefore, the beam can be oriented in a perpendicular path relative to the stent tubing.

For example, as shown in FIG. 5, when minor 210 is angled at position 1, while the laser beam exits telecentric F-theta lens 215 perpendicularly to the lens, the laser beam is not oriented directly in line with the top dead center of stent tube 220. Because of this misalignment, the laser beam is not focused directly on the top dead center of the stent tube, and impinges the stent tube at location 225. By simply changing the angulation of minor 210 to position 2, the location of the focal point of the exiting laser beam can be adjusted to impinge the stent tube at location 230, which in this example is the desired top dead center of the stent tube. Note that the laser beam still exits telecentric F-theta lens 215 perpendicularly to the lens. This greatly simplifies the adjustment and alignment of the laser system.

The focused laser beam exiting a telecentric lens always strikes a working field at an angle normal to the surface of the working field. In contrast, the focused beam of a non-telecentric lens strikes the working field at greater and greater angles of incidence as the beam travels farther and farther from the center of the field. An example of this is seen in FIG. 6, where light beam 370 falls upon a minor 375 which has angulation 375b. In this case, laser beam 382 exits lens 380 perpendicular to the lens, because laser beam 370 is reflected so that it is parallel to the central axis of lens 380. If mirror 375 is placed at position 375a, laser beam 370 not enters lens 380 off axis, and the focal point of exit beam 384 is displaced and the beam is focused above plane 386.

FIG. 7 is an illustration of the effect of using an F-theta lens 385 in place of lens 380. An F-theta lens also exhibits unique properties that are useful to this invention. An F-theta lens is capable of providing a flat image field at the plane of interest. This means that when light beam 370 is reflected by minor 375 when minor 375 is angled in position 375a, and impinges upon F-theta lens 385, light beam 389 is displaced from the intended focus point, but the focal point of beam 389 is on plane 386. While it would be possible, in some cases, to substitute an F-theta lens in the optical chain to allow the positioning of the focal point on plane 386 to be adjusted by adjusting mirror 375, because beam 389 does not exit lens 389 perpendicularly to the lens axis, beam 389 impinges on plane 386 at an angle, which can result in a non-point focus condition of the laser spot resulting in imprecise cutting and reduced cutting speed, among other potential problems.

Combining the unique properties of a telecentric lens and an F-theta lens results in an advantageous lens design, which is illustrated in FIG. 8. The telecentric F-theta lens 390 produces a cutting beam that is perpendicular to a cutting plane and that has a focal point intersecting with the cutting plane regardless of the angle at which the laser beam enters the lens. The telecentric F-theta lens 390 is an improvement over the ordinary F-theta lens 385 (FIG. 9) in this respect, which is clear when comparing the exit angles of the laser beam 370 when mirror 375 is positioned at angle 375a and angle 375b. In both cases, light beams 392 and 394 exit telecentric F-theta lens 390 at an angle perpendicular to the central axis of lens 390, thus ensuring that the focal point of the laser beam is optimally focused, event thought the position of the beam on surface 386 has been shifted.

By using this type of lens in the optical chain of laser cutting equipment, there are several primary benefits. First, since the beam will always be perpendicular to the target material, the plane of the stent strut will tend to be perpendicular to the outer surface of the stent tubing. Second, if the beam becomes slightly mal-positioned relative to the top-dead-center position, it may be brought back into position easily by changing the angle of the minor without the need to adjust the alignment of other components of the laser optical chain. Finally, the focal point of the laser beam will coincide with the same cutting plane regardless of the angle that the beam is directed through the telecentric F-theta lens.

In another embodiment, the present invention includes a detector that can be used to calibrate the position of a laser beam spot on the stent tube. The detector provides a signal to a processor that utilizes the signal to adjust the position of a minor 210 (FIG. 5) or the compensation components 450, 410 (FIG. 9) to correctly position the beam spot at a desired location to effect cutting of a stent pattern into the stent tube. In this embodiment, a reflective calibration surface is used to reflect a laser beam away from the cutting surface toward a detector. Based on the detector measurement, the beam position and alignment can be easily ascertained. Furthermore, if the beam is out of alignment, a parallel plate may be used to shift the laser beam as it is directed through a lens in order to laterally shift the cutting spot. In an alternative embodiment, a Risley prism pair may be used to correct angular shifts of the laser beam as it travels through the optical chain. Accordingly, the use of the various embodiments of the invention provides a relatively easy means to monitor laser beam position and to adjust for any lateral or angular shifts in the laser beam that are detected.

Referring now to FIG. 9, a system 400 for monitoring the position of a laser beam relative a target material, such as a stent tube is shown. A laser beam 405 is directed toward a partially reflective beam splitting mirror 410. Minor 410 allows the laser beam to pass through but reflects a very small portion of the laser beam to a pair of detectors 415, 420 positioned between a focusing lens 425 and mirror 410. The detectors 415, 420 may have several configurations that are well known in the art. In one embodiment, for example, the detectors may be a CCD matrix that can detect the incoming laser light and compare it to the desired beam position.

In one embodiment the desired beam position is determined through a calibration procedure in which the laser beam spot is positioned on the cutting spot 430 of stent tube 435 where the operator would like it to be and then the beam is reflected away from the cutting part toward the detector 420 through the lens 425 and the mirror 410, with position of the incoming beam being recorded on detector 415.

Reflection of the beam away from the cutting part can be enhanced by placing a reflective surface 440 adjacent to the stent tube 435 so that the incoming laser beam is reflected rather than reaching the stent tube 435. After this calibration procedure, the position of the laser on the work piece can be ascertained at any time by reinserting the reflective surface 440 and detecting the position of the reflected beam on detector 420. If the relationship of the beam positions on detector 415 and detector 420 differs from the as-calibrated position, then the operator may be notified that the beam cutting position has changed and requires repositioning and/or realignment.

In another embodiment, additional optical components may be added to the optical chain that allow an operator to quickly and effectively modify both the laser beam position and alignment. Shifting the beam alignment is enabled by the introduction of a parallel plate 445 into the optical chain as shown in FIG. 9. The parallel plate diffracts incoming light such that the exiting light is shifted laterally. By altering the angle of the parallel plate, the lateral shift will vary as well. For example, if the parallel plate is positioned such that incoming light is perpendicular to the plate, then the outgoing light will be perfectly aligned with the incoming light.

Alternatively, the more that the parallel plate is angled, the greater the lateral shift of the incoming light. By using one or more of these parallel plates positioned in different locations and arranged to shift the light in different planes, the lateral position of the light as it enters the focusing lens can be effectively controlled. Therefore, shifts of the focal point of the laser at the stent tube can be quickly compensated for by altering the parallel plate orientation.

In addition to controlling the lateral position of the laser beam, an alternative embodiment compensates for angular shifts of the laser that may occur during use. This angular shift compensation is accomplished by integrating a Risley prism pair 450 in the optical chain. The Risley prism pair allows the angular shift between incoming and outgoing light to be controlled simply by rotating the prisms relative to each other. When both of the prisms refract light in the same direction, the pair acts as a single prism with twice the prism angle of either prism alone. Therefore, if the incoming light enters the first prism at a different than desired angle, the relative orientation of the two prisms can be changed to compensate for this angle and ensure that the exiting light is directed perpendicular to the next component of the optical chain. The Risley prism pair may introduce an unwanted lateral shift which can be compensated by the parallel plate 445.

By combining the position calibration technique and the lateral and angular compensation components as described above, the various embodiments of the invention allow for the laser beam cutting position to be easily monitored and maintained. This represents a significant improvement in control and overall process efficiency as compared to previous methods of accomplishing these tasks. Moreover, the various embodiments of the system and methods of the invention eliminate the need to perform a stage-assistant alignment between the laser beam and the stent tube, which significantly improves manufacturing efficiency. Another advantage is that the various embodiments of the present invention also provide the ability to automate beam position correction.

Other modifications and improvements may be made without departing from the scope of the invention. Accordingly, it is not intended that the invention be limited, except as by the appended claims.

I claim:

1. A laser system for cutting a stent pattern into a stent tube, comprising:
a laser for generating a laser beam;
a mirror for reflecting the laser beam at a desired angle; and
a telecentric F-theta lens configured to receive the reflected laser beam and to focus the reflected laser beam onto a stent tube.

2. The laser system of claim 1, further comprising:
a partially reflecting and partially transmitting minor disposed in the laser beam between the mirror and the telecentric F-theta lens;
a first detector disposed to receive a portion of light from the incoming laser beam reflected by the partially reflecting and partially transmitting mirror, the first detector configured to provide a signal representative of the position of the incoming laser beam on the first detector;
a second detector disposed to receive a portion of light from a laser beam reflected from the stent tube and subsequently reflected by the partially reflecting and partially transmitting minor, the second detector configured to provide a signal representative of the position of the reflected laser beam on the second detector.

3. The laser system of claim 2, further comprising:
a reflective surface disposed between the telecentric F-theta lens and the stent tube.

4. The laser system of claim 2, further comprising:
a parallel plate disposed in the path of the incoming laser beam before the partially reflecting and partially transmitting mirror.

5. The laser system of claim 2, further comprising:
a Risley prism pair disposed in the path of the incoming laser beam before the partially reflecting and partially transmitting mirror.

6. A method for aligning the focal spot of a laser beam used to cut a stent pattern into a stent tube, comprising:
disposing a tiltable minor into a laser beam between a laser and a stent tube;
disposing a telecentric F-theta lens into the laser beam between the tiltable mirror and the stent tube;
focusing the laser beam to form a focused laser spot on the stent tube; and
tiling the mirror to adjust the position of the focused laser spot on the stent tube.

7. A method for compensating for lateral or angular shift of a laser beam focused on a stent tube, comprising:
disposing a partially reflecting and partially transmitting minor between a laser generating a laser beam and focusing lens, the focusing lens configured to focus the laser beam at a location on the stent tube;
disposing a first detector in a position to receive a portion of light from the incoming laser beam reflected by the partially reflecting and partially transmitting mirror, the first detector configured to provide a signal representative of the position of the incoming laser beam on the first detector;
disposing a second detector in a position to receive a portion of light from a laser beam reflected from the stent tube and subsequently reflected by the partially reflecting and partially transmitting minor, the second detector configured to provide a signal representative of the position of the reflected laser beam on the second detector; and
comparing the signals representing the positions of the incoming laser beam and the reflected laser beam to determine if the position of the incoming laser beam on the stent tube has changed.

8. The method of claim 7, further comprising:
disposing a parallel plate into the laser beam prior to the partially reflecting and partially transmitting minor; and
arranging the parallel plate to laterally shift the position of the incoming laser beam on the stent tube.

9. The method of claim 7, further comprising:
disposing a Risley prism pair into the laser beam prior to the partially reflecting and partially transmitting minor; and
arranging the Risley prism pair to shift the angular position of the incoming laser beam on the stent tube.

10. The method of claim 9, further comprising:
disposing a parallel plate into the laser beam after the Risley prism pair and before the partially reflecting and partially transmitting mirror; and
arranging the parallel plate to compensate a lateral shift in the position of the incoming laser beam introduced by the Risley prism pair.
